(19) United States
(12) Patent Application Publication
(10) Pub. No.: US 2025/0154557 A1
(43) Pub. Date: May 15, 2025

(54) COMPOSITION FOR SELECTING NUCLEIC ACIDS OF INTEREST, AND A METHOD FOR SELECTING NUCLEIC ACIDS USING THEREOF

(71) Applicants: GWANGJU INSTITUTE OF SCIENCE AND TECHNOLOGY, Gwangju (KR); Seoul National University R&DB Foundation, Seoul (KR)

(72) Inventors: Yeongjae Choi, Gwangju (KR); Sunghoon Kwon, Seoul (KR); Yoon Hae Koh, Gwangju (KR); Woo Jin Kim, Gwangju (KR); Mingweon Chon, Seoul (KR); Hansol Choi, Seoul (KR)

(21) Appl. No.: 18/932,174

(22) Filed: Oct. 30, 2024

(30) Foreign Application Priority Data
Nov. 15, 2023 (KR) 10-2023-0158172

(51) Int. Cl. C12Q 1/6806; C12Q 1/6869

(52) U.S. Cl. CPC C12Q 1/6806; C12Q 1/6869

(57) ABSTRACT

The polymerase chain reaction (PCR) has limitations, including a lack of efficient search strategies and inefficiencies in securing primer sequence lengths, despite its high sensitivity and specificity. Therefore, the present invention has been devised to address these issues, concerning a composition for the selection of the desired nucleic acid and a method for nucleic acid selection using it. By using the composition and method of the present invention, it becomes possible to hierarchically, efficiently, and selectively detect and amplify subsets of oligonucleotides with high specificity, which is expected to be widely utilized in the overall bio/medical field.

The present invention was undertaken with the supported by the Pioneer Research Center Program through the National Research Foundation of Korea funded by the Ministry of Science, ICT & Future Planning (NRF-2022M3C1A3081366), and the Ministry of Science and ICT (MSIT) of the Republic of Korea and the National Research Foundation of Korea (NRF-2020R1A3B3079653, 2022R1C1C1010938).

### CROSS-REFERENCE TO RELATED APPLICATIONS

This application claims the benefit of Korean Patent Application no. 10-2023-0158172, filed Nov. 15, 2023, which is hereby incorporated herein by reference in its entirety.

### BACKGROUND

### 1. Technical Field

The present invention relates to composition for selecting nucleic acids of interest, and a method for selecting nucleic acids using thereof.

### 2. Related Art

Polymerase chain reaction (PCR) is one of the most widely adopted methods for selectively amplifying target DNA recognized by primer sequences. PCR has enabled the selective amplification of target regions for amplification from complex oligonucleotides with millions of different sequences.

However, despite the high sensitivity and specificity of PCR, two main limitations still remain in PCR-based selective amplification. First, PCR lacks an efficient search strategy. PCR is an on/off selection that can only detect the presence or absence of target DNA with specific primer sequences. Multiplex PCR can amplify 384 sets of targets using different primer sequences in a single reaction, providing scalability, but the overall sequencing rate of targets within the total reads is only 43%. Additionally, there is a lack of methods that can hierarchically search multiple oligo subsets. Second, there is inefficiency in securing primer sequence length. Each subset targeted for amplification requires different primer sequences, and about 40 nucleotides (nt) are needed in the primer region to secure specificity between sequences. However, since the synthesis limit of oligonucleotides is about 200 nt, allocating a 40 nt region to primers poses a very inefficient problem. Moreover, when synthesizing oligos using microarray-based technology, the amount of synthesized oligo is typically as low as 1 picomole, so universal primer regions are generally assigned to both ends to amplify the entire oligo library. Assuming that both universal primer regions and selective primer regions are introduced, even if only two different oligo subsets are selected, a total of 80 nt of primer regions are needed for a 200 nt oligo. Therefore, there is a need for the development of techniques that can hierarchically manage subsets of oligos and search oligo subsets while minimizing nucleotide allocation for precise applications.

Thus, the present invention has been conceived to solve the aforementioned problems, relating to a selective composition for the desired nucleic acid and a method for selecting nucleic acids using it. Utilizing the composition and method of the present invention enables the hierarchical, efficient, and selectively specific detection and expansion of oligo subsets, and is expected to be widely used in the overall bio/medical field.

### SUMMARY

An object of the present invention is to provide a method for selecting the target nucleic acid. Another object of the present invention is to provide a method for elongating the target nucleic acid. Another object of the present invention is to provide a composition for selecting a target nucleic acid. Another object of the present invention is to provide a composition for elongating a target nucleic acid. Another object of the present invention is to provide a kit for selecting a target nucleic acid. Another object of the present invention is to provide a kit for elongating a target nucleic acid.

However, objects of the present invention are not limited to the objects mentioned above, and other objects not mentioned herein may be clearly understood by those of ordinary skill in the art from the following description.

Hereinafter, various embodiments described herein will be described with reference to figures. In the following description, numerous specific details are set forth, such as specific configurations, compositions, and processes, etc., in order to provide a thorough understanding of the present invention. However, certain embodiments may be practiced without one or more of these specific details, or in combination with other known methods and configurations. In other instances, known processes and preparation techniques have not been described in particular detail in order to not unnecessarily obscure the present invention. Reference throughout this specification to “one embodiment” or “an embodiment” means that a particular feature, configuration, composition, or characteristic described in connection with the embodiment is included in at least one embodiment of the present invention. Thus, the appearances of the phrase “in one embodiment” or “an embodiment” in various places throughout this specification are not necessarily referring to the same embodiment of the present invention. Additionally, the particular features, configurations, compositions, or characteristics may be combined in any suitable manner in one or more embodiments.

Unless otherwise stated in the specification, all the scientific and technical terms used in the specification have the same meanings as commonly understood by those skilled in the technical field to which the present invention pertains.

Polymerase chain reaction (PCR) is one of the most widely adopted methods for selectively amplifying target DNA recognized by primer sequences. PCR has enabled the selective amplification of target regions for amplification from complex oligonucleotides with millions of different sequences. However, despite the high sensitivity and specificity of PCR, two main limitations still remain in PCR-based selective amplification. First, PCR lacks an efficient search strategy. PCR is an on/off selection that can only detect the presence or absence of target DNA with specific primer sequences. Multiplex PCR can amplify 384 sets of targets using different primer sequences in a single reaction, providing scalability, but the overall sequencing rate of targets within the total reads is only 43%. Additionally, there is a lack of methods that can hierarchically search multiple oligo subsets. Second, there is inefficiency in securing primer sequence length. Each subset targeted for amplification requires different primer sequences, and about 40 nucleotides (nt) are needed in the primer region to secure specificity between sequences. However, since the synthesis limit of oligonucleotides is about 200 nt, allocating a 40 nt region to primers poses a very inefficient problem. Moreover, when synthesizing oligos using microarray-based technology, the amount of synthesized oligo is typically as low as 1 picomole, so universal primer regions are generally assigned to both ends to amplify the entire oligo library. Assuming that both universal primer regions and selective primer regions are introduced, even if only two different oligo subsets are selected, a total of 80 nt of primer regions are needed for a 200 nt oligo. Therefore, there is a need for the development of techniques that can hierarchically manage subsets of oligos and search oligo subsets while minimizing nucleotide allocation for precise applications. Thus, the present invention provides a composition for the selective amplification of target nucleic acids and an amplification method using the same to solve the aforementioned problems.

In one aspect of the present invention, a method for selecting the target nucleic acid is it provided.

The researchers believe that introducing the concept of directories, commonly used for managing digital data in each oligo subset, could resolve the main disadvantages of PCR. In computer science, data is managed as files with their own directories for retrieval, allowing for hierarchical data structures and easy access to multiple files. For example, files can be retrieved or accessed by sequentially specifying subdirectories to parent directories, and multiple files sharing a subdirectory can be selected simultaneously by folder. However, such oligo library management systems have not yet been attempted due to the lack of methods for recognizing several oligo sequences to distinguish different oligos. However, recent developments in next-generation sequencing (NGS) have enabled the identification of all nucleotides of oligos with single nucleotide resolution. NGS involves introducing a reversible terminator complementary to the template strand sequence of DNA, allowing for the addition of one nucleotide at a time. Therefore, it is expected that the combination of the directory system in computer science and the nucleotide identification technology of NGS can efficiently manage and retrieve oligo subsets.

In this invention, the researchers propose oligo subset selection technology by introducing a quaternary directory for each oligo subset. The quaternary directory consists of several levels similar to hierarchical folders in computer science, with each level comprising four types of nucleotides (adenine, thymine, guanine, and cytosine) (FIG. 1A). The proposed technology can search oligo subsets by recognizing nucleotide types at each level of the directory, similar to binary search in computer science. The greatest advantage of the introduced method is that the directory levels can be expanded based on the number of oligo subsets. For example, a 4-level directory for 2 nt can encode up to 16 (4²) different DNA subsets, while PCR-based selective amplification requires a minimum of 40-nt primer regions. Additionally, this technology allows for the efficient search of multiple oligo subsets through its hierarchical structure. For oligo subsets with a 4 bp directory, if adenine and thymine nucleotides are combined with a reversible terminator and the search is stopped in the second cycle, all oligo subsets matching the first and second directories are retrieved regardless of the remaining directory (FIG. 1A).

The search begins by hydrogen bonding specific types of nucleotides with reversible terminators matching the target directory (e.g., 3′-O-azidomethyl deoxynucleotide), while other types of nucleotide's bond with irreversible terminators (e.g., dideoxynucleotide) (FIG. 1B). The original DNA library is fixed to a solid substrate, such as magnetic beads, making it easy to replace the formulation used in the selection reaction. The selection process starts with the hybridization of primers complementary to a universal primer region, and the selection cycle consists of three stages. First, various combinations of bases with reversible terminators and bases with irreversible terminators are introduced. At the last stage of the cycle, the blocker of the reversible terminator is removed, allowing binding in subsequent cycles. The search is repeated by removing the blocker of the reversible terminator until the desired directory level is reached. After that, only the target oligo with the reversible terminator can recover the full DNA sequence through length extension. Selective elongation is activated for the target oligo from which the terminator has been removed after the last cycle. Oligos bound to irreversible terminators that do not match the target directory cannot elongate. Oligos that have completed length extension to the full DNA sequence are distinguishable by their length from the remaining oligo fragments. Additionally, using the universal primer regions at both ends, only fully extended oligos can be amplified by PCR. As a result, the proposed sequential process allows for the selection of a desired subset of oligos from a complex oligo pool.

To implement this as a process, the method of selecting the target nucleic acid of the present invention may be comprises a step of: (a) identifying a part of the nucleotide sequence of the target nucleic acid as XₙXₙ₊₁; (b) treating a mixture composed of two or more types of base units to target nucleic acid Xₙ; (c) removing the bases mixture from step (b); and (d) recognizing the base units bound to the target nucleic acid in step (b).

In the method, n may be a natural number.

The mixture composed of two or more types of base units in step (b) may be a mixture of bases with reversible terminators and bases with irreversible terminators, may be a mixture of unlabeled dNTPs and bases with reversible terminators, may be a mixture of unlabeled dNTPs and bases with irreversible terminators, or may be a mixture of unlabeled dNTPs, bases with reversible terminators and bases with irreversible terminators, but is not limited to these.

The method for selecting the target nucleic acid of the present invention may also further include, after step (d), a step of (e) polymerizing bases to bind only to the target nucleic acid using a polymerase.

In the present invention, the term “reversible” means that it is possible to return to the original state, while the term “irreversible” means that it is impossible to return to the original state. In this invention, in order to amplify the target nucleic acid, it is prerequisite to identify the nucleotide sequence of the corresponding nucleic acid, and based on this, it is assumed to provide a combination of nucleotides that can complementarily bind to the sequence of the target nucleic acid. At this time, the nucleotide combination may be a mixture of nucleotides with reversible terminators or with irreversible terminators. Here, the reversible or irreversible terminators can be interpreted as labeling, tagging or marking. Both the nucleotides with reversible terminators or with irreversible terminators can perform the function of blocking the connection of a second nucleotide after the first nucleotide; however, in the case of the nucleotides with reversible terminators, the terminators can be removed or modified according to a suitable process, and in such cases, additional nucleotides can be connected afterward.

In the method for selecting the target nucleic acid of the present invention, the reversible terminator may be selected from the group consisting of an azidomethyl moiety, an allyl moiety, and a nitrobenzyl moiety. If the reversible terminator is an azidomethyl moiety, the removing the blocker of the reversible terminator may be performed using tris(2-carboxyethyl) phosphine. If the reversible terminator is an allyl moiety, the removing the blocker of the reversible terminator may be performed using sodium tetrachloropalladate, or sodium triphenylphosphine trisulfonate. If the reversible terminator is a nitrobenzyl moiety, the removing the blocker of the reversible terminator may be performed by laser irradiation of 345 to 365 nm, but this is not limited to those methods. Moreover, the bases with irreversible terminators may be a dideoxynucleotide (ddNTP).

In the present invention, the term “nucleic acid” refers to a polymer substance in the form of a long nucleotide chain composed of nucleotides consisting of a base, pentose, and phosphate group, connected by phosphodiester bonds. It is a substance that governs heredity and protein synthesis, serving as a blueprint for the genetic information of life. It is distinguished as RNA (RiboNucleic Acid) when the pentose is ribose, and DNA (Deoxyribo Nucleic Acid) when it is deoxyribose.

In the present invention, the term “base” refers to the nitrogenous bases that make up the nucleic acid, which is a molecule containing one or two rings composed of carbon and nitrogen atoms. This molecule is called a “base” because it is chemically basic and can bond with hydrogen ions. There are two types of nitrogenous bases: pyrimidines and purines. Pyrimidines consist of a heterocyclic ring with six atoms, including two nitrogen atoms, and include cytosine (C), thymine (T), and uracil (U). Purines have a two-ring structure formed by the fusion of a pyrimidine ring and an imidazole ring, including adenine (A) and guanine (G). Cytosine, adenine, and guanine are present in both DNA and RNA, while thymine is only in DNA, and uracil is only in RNA. Purines and pyrimidines can form hydrogen bonds in a complementary pattern, similar to puzzle pieces. Under typical cellular conditions, adenine forms hydrogen bonds with thymine in DNA or uracil in RNA, while guanine forms hydrogen bonds with cytosine. This is referred to as complementary.

In the method for selecting the target nucleic acid according to the present invention, the base is selected from the group consisting of adenine, thymine, cytosine, guanine, isoguanine, isocytosine, 2-amino-6-(2-thienyl)purine, pyridine-2-one, pyrrole-2-carbaldehyde, 7-(2-thienyl)imidazo[4,5-b]pyridine, 2,6-dimethyl-2H-isoquinoline-1-thione, 2-Methoxy-3-methylnaphthalene, 2-amino-imidazo[1,2-a]-1,3,5-triazin-4(8H)one, 6-amino-5-nitro-2(1H)-pyridone, 7-(2,2′-bithien-5-yl)-imidazo[4,5-b]pyridine, 4-[3-(6-aminohexanamido)-1-propynyl]-2-nitropyrrole, and inosine, but are not limited to these.

In the present invention, a method for selecting a target nucleic acid using a base combination that includes a mixture of bases with reversible terminators and bases with irreversible terminators that can complementarily bind to the sequence of the target nucleic acid is exemplified. When the target nucleic acid region consists of two bases (X₁X₂), wherein X₁ is cytosine (C) and X₂ is guanine (G), the first base combination may be a mixture of 3′-O-azidomethyl dGTP as bases with reversible terminators for the complementary base to cytosine, and ddATP, ddTTP, and ddCTP as bases with irreversible terminators for the non-complementary bases to cytosine. Additionally, the second base combination may be a mixture of 3′-O-azidomethyl dCTP as bases with reversible terminators for the complementary base to guanine, and ddATP, ddTTP, and ddGTP as bases with irreversible terminators for the non-complementary bases to guanine. After contacting with the first base combination, 3′-O-azidomethyl dGTP binds to cytosine (C), which is X₁. The 3′-O-azidomethyl functions as a blocking agent that prevents X₂, guanine (G), from binding with other complementary bases, but can be removed by treatment with tris(2-carboxyethyl)phosphine hydrochloride (TCEP). In this case, additional bases can be connected, so in the subsequently contacted second base combination, 3′-O-azidomethyl dCTP binds to guanine (G), which is X₂.

In another aspect of the present invention, a more specific method for selecting the target nucleic acid is provided.

The more specific method of selecting the target nucleic acid of the present invention may be comprises a step of: (a) identifying a part of the nucleotide sequence of the target nucleic acid as XₙXₙ₊₁; (b) treating a mixture composed of complementary bases with reversible terminators and non-complementary bases with irreversible terminators to target nucleic acid Xₙ; (c) removing the bases mixture from step (b); and (d) removing the blocker of the reversible terminator from step (b).

In the method, n may be a natural number.

The method for selecting the target nucleic acid of the present invention may also further include, after step (d), (e) treating a mixture composed of complementary bases with reversible terminators and non-complementary bases with irreversible terminators to target nucleic acid Xₙ₊₁; (f) removing the bases mixture from step (e); and (g) removing the blocker of the reversible terminator from step (e).

Additionally, the method for selecting the target nucleic acid of the present invention may also further include, after step (a), (a-1) adding a primer that recognizes the polynucleotide sequence of the target nucleic acid.

In the more specific method for selecting the intended nucleic acid of the present invention, the specific definitions of reversible terminators, irreversible terminators, nucleic acids, and bases are omitted to avoid complexity in the specification, as they overlap with those described above.

In another aspect of the present invention, a method for elongating the target nucleic acid is provided.

In the method for elongating the target nucleic acid of the present invention, the method can include the aforementioned method for selecting the target nucleic acid. Specifically, it comprising a step of: (a) identifying a part of the nucleotide sequence of the target nucleic acid as XₙXₙ₊₁; (b) treating a mixture composed of complementary bases with reversible terminators and non-complementary bases with irreversible terminators to target nucleic acid Xₙ; (c) removing the bases mixture from step (b); (d) removing the blocker of the reversible terminator from step (b); (e) treating a mixture composed of complementary bases with reversible terminators and non-complementary bases with irreversible terminators to target nucleic acid Xₙ₊₁; (f) removing the bases mixture from step (e); and (g) removing the blocker of the reversible terminator from step (e); wherein the steps (e) to (g) are repeated to elongate the target nucleic acid region.

In another aspect of the present invention, compositions for selecting or elongating a target nucleic acid are provided.

The composition of the present invention may be a mixture composed of complementary bases with reversible terminators and non-complementary bases with irreversible terminators to target nucleic acid, which may include dATP with reversible terminators, ddTTP, ddCTP, and ddGTP; dTTP with reversible terminators, ddATP, ddCTP, and ddGTP; dCTP with reversible terminators, ddATP, ddTTP, and ddGTP; or dGTP with reversible terminators, ddATP, ddTTP, and ddCTP. And in above composition, wherein the reversible terminator may be selected from the group consisting of an azidomethyl moiety, an allyl moiety, and a nitrobenzyl moiety.

In another aspect of the present invention, a kit for selecting or elongating a target nucleic acid is provided.

The kit may include the composition for selecting or elongating a target nucleic acid as described above.

The method for selecting a target nucleic acid, the method for elongating the target nucleic acid, and the compositions/kits for implementing these may be used in genetic diagnosis and other applications.

The effects of the present invention are as follows. The compositions and methods of the present invention allow for the hierarchical, efficient, selective, and highly specific detection and elongation of subsets of oligos, and are therefore expected to be of great use in the overall bio/medical field.

### BRIEF DESCRIPTION OF THE DRAWINGS

FIGS. 1A-1B show the synthesis and selection-based oligo selection enables 4N subsets access with N nucleotide region and a hierarchical structure in complex oligo library according to the present invention. (FIG. 1A) Oligo library subset selection employing a hierarchical structure. Accessing a higher level of the hierarchy enables reaching all the oligo subsets below it. The DNA sequence-based barcoding system utilizes four base types and employs a quaternary barcode-based method for representing directories. By contrast, traditional PCR-based oligo selection requires a one-to-one mapping of specific primers to oligo subsets. The hierarchical structure within the oligo library demonstrates scalability through reduced nucleotide length requirements and improved programmability in oligo selection. (FIG. 1B) Details of the oligo selection include single-base resolution synthesis and selection cycles on the complementary template oligo strands. During synthesis and selection, nucleotides matching the target directory are introduced with reversible terminators, such as 3′-O-azidomethyl deoxynucleotides, while others are coupled with irreversible terminators, such as dideoxynucleotides. By repeating the cycles after deprotecting the irreversible terminators, only the oligos with target barcodes will be retrieved.

FIGS. 2A-2D show multiple modes of oligo subset selection that were enabled by synthesis and selection along with a hierarchical barcode system according to the present invention. (FIG. 2A) Five oligos of varying lengths with identical universal primer were designed and mixed at equal molar concentrations before selection. A 4 nt barcode was assigned to demonstrate various selection methods. (FIG. 2B) Single, (FIG. 2F) hierarchical, and (FIG. 2D) multiple selections of oligo subsets were demonstrated. The hierarchical structure of the barcodes for the five oligos is depicted on the left, where selecting a barcode at a higher level enables simultaneous selection of all connected lower-level barcodes, akin to accessing files within a folder. Barcodes highlighted in cyan are examples used for selection, with the corresponding selected oligos illustrated next to them. The results of polyacrylamide gel electrophoresis after selection are displayed, listing the selected barcodes in sequence below the image.

FIGS. 3A-3E show scalable, programmable oligo subset selection within a hierarchically structured complex oligo library according to the present invention. (FIG. 3A) The complex oligo library is designed to accommodate the hierarchical organization of barcodes. The library encodes data from four classical music pieces totaling 96.88 kB, spread across 12,000 oligos. Each oligo is 200 bp long, with 4 nt for the barcode. Barcodes are hierarchical; the first two nucleotides (N1, N2) differentiate pieces and instruments, while the next two (N3, N4) address specific sections. (FIG. 3B) Two nt and (FIG. 3C) 4 nt hierarchical barcode-based selection were demonstrated and analyzed using NGS. Read counts for each designed oligo were normalized and plotted in bar graphs. The left graph shows distribution before selection; the right shows results after. The red dashed line indicates the normalized read count, averaged at one. Pie charts show the proportion of total read counts, with blue for target barcodes before selection, green for target barcodes after, and gray for non-targets. The 2 nt barcode reveals the library's hierarchical structure. The barcode ‘TG’ targets the trombone part of Mozart's Requiem in D minor. Selecting a 4 nt barcode decodes specific musical segments. The barcode ‘ATAC’ targets a segment of Pachelbel's Canon in D Major Viola part. (FIG. 3D, and FIG. 3E) Multiple selection scenarios within the oligo library, with graphs representing selection to 1, 2, and 3 nt barcodes from the left, and the lower graph for a 4 nt barcode. Two barcodes at each length were selected simultaneously, marked in cyan on the tree above each graph.

FIGS. 4A-4E show selection efficiency of multiple cycles of selection and rare population of oligo subset according to the present invention. (FIG. 4A) The read count ratio for every synthesis and selection cycle of 4 nt barcodes was analyzed using 10 distinct barcodes. (FIG. 4B) The Enrichment fold (EF) values of the barcodes showed no significant bias between each barcode; the dotted line represents the average of each EF. (FIG. 4C) The ratio of selected target sequences by sequencing coverage. Results are compared with PCR-based selection. (FIG. 4D) Demonstration of subset selection within the rare population of an oligo library. The read count ratios for each cycle of four distinct 8 nt barcode selections were analyzed. The blue graph illustrates the ratio for one barcode assigned to three oligo designs, and the green graph illustrates the ratio for three barcodes each assigned to one designed strand, out of a diversity of 12,000. (FIG. 4E) The distribution of read counts per million of all barcodes was analyzed. The left graph shows the distribution before selection, and the right graph shows the distribution after selection. The ratio of target barcodes increased by an average of 57.855 times following the selection process.

FIGS. 5A-5C show a schematic demonstrating subset replacement through negative synthesis and selection in a hierarchically structured oligo library according to the present invention. (FIG. 5A) Each layer of the barcode sequences corresponds to different levels of data, from musical pieces to specific instrument sections. The process represents the replacement from the original data (contrabass #5) to new data (violin #5) through the negative synthesis and selection. (FIG. 5B) The negative synthesis and selection process followed by file replacement involves blocking the target barcode while allowing selection of the others. Subsequently, a newly synthesized oligo subset is added to replace the file. (FIG. 5C) To verify the target file replacement, we plotted the normalized read counts from various barcode selection scenarios in a bar graph.

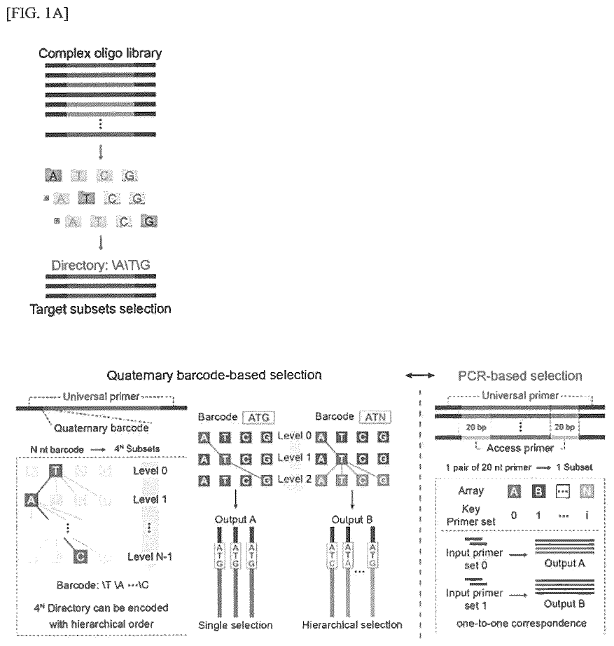

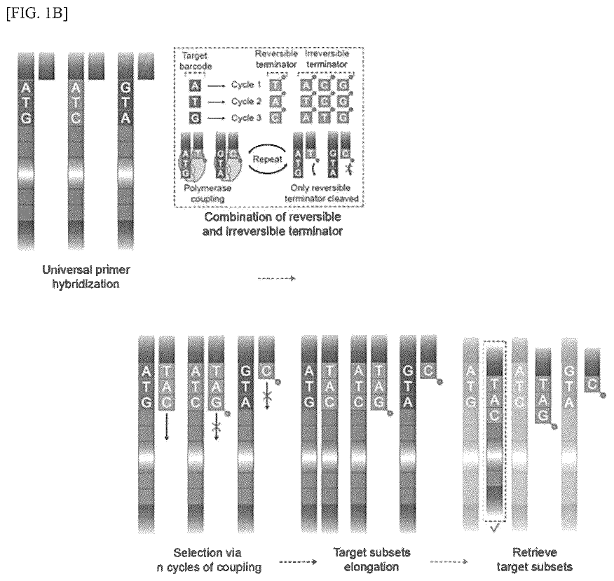

### DETAILED DESCRIPTION

Hereinafter, the present invention will be described in more detail with reference to examples. These examples are only for illustrating the present invention in more detail, and it will be apparent to those of ordinary skill in the art that the scope of the present invention according to the subject matter of the present invention is not limited by these examples.

### Example

### Methods

### 1. Process of Synthesis and Selection-Based Selection

The synthesis and selection cycle involved introducing 3′-O-azidomethyl-dNTPs complementary to the barcode, while ddNTPs excluding the complementary base were added. Tris(2-carboxyethyl) phosphine (TCEP) was employed to cleave the azidomethyl groups. After each coupling and cleavage step, the beads were washed three times with 1× ThermoPol® Reaction Buffer.

For the coupling step, a mixture containing 1 μL of 1 mM 3′-O-azidomethyl-dNTP, 1 μL each of 2 mM ddNTP, 5 μL of ThermoPol® Reaction Buffer, 1 μL of Therminator™ III DNA Polymerase, and 40 μL of nuclease-free water was incubated at 65° C. for 30 seconds. Cleavage was executed by treating the beads with 50 μL of 100 mM pH 9.0 TCEP at 65° C. for 1 minute.

Following the final cycle, the beads were treated with Bst DNA Polymerase to elongate the dNTPs and washed three times with 1× ThermoPol® Reaction Buffer. The beads were then treated with 50 μL of 8 mM urea and incubated at 70° C. for 3 minutes to ensure denaturation. The supernatant separated from the beads was purified using Monarch® Nucleic Acid Purification Kits (New England Biolabs) to complete the subset selection process.

### 2. Immobilization of Oligo Library on Magnetic Beads

To amplify the oligonucleotides, a forward primer (ACACTCTTTCCCTACACGACGCTCTTCCGATCT) and a reverse primer (GTGACTGGAGTTCAGACGTGTGCTCTTCCGATCT) were used. The reaction mixture, comprising 2 μL of each 10 μM primer, 0.2 μL of AccuPrime™ Taq DNA Polymerase (Thermo Scientific™), 5 μL of AccuPrime™ PCR Buffer I, 2 μL of template DNA (1.17 ng/L), and 38.8 μL of nuclease-free water, was incubated with the following protocol: (1) initial denaturation at 94° C. for 15 s, (2) denaturation at 94° C. for 15 s, (3) annealing at 58° C. for 15 s, (4) extension at 68° C. for 30 s, with steps 2-4 repeated for 11 cycles for five oligos and 24 cycles for oligo library. The amplicons were stored at −20° C. before use.

The amine-modified reverse primer was immobilized onto magnetic beads coated with N-hydroxysuccinimide (NHS) ester reactive groups (Thermo Scientific™). To anneal the amplified oligonucleotide to the primer on the bead, the mixture underwent denaturation at 95° C. for 30 seconds, followed by a gradual cooling from 95° C. to 65° C. at a rate of 1° C. per 30 seconds. Following annealing, the beads were washed three times with 1× ThermoPol® Reaction Buffer. the extension phase involved adding 1 μL of Bst DNA Polymerase (New England Biolabs), 3 μL of 100 mM Magnesium Sulfate (MgSO4) Solution, 5 μL of ThermoPol® Reaction Buffer, 1 μL of 10 mM dNTP, and 40 μL of nuclease-free water. The mixture was then incubated at 65° C. for 1 minute. The beads were washed three times with 1× ThermoPol® Reaction Buffer.

To prepare for single-stranded DNA selection, 50 μL of 8 mM urea was added to the beads with double-stranded DNA. This mixture was denatured at 70° C. for 3 minutes and washed three times with 1× ThermoPol® Reaction Buffer to retain only the single-stranded DNA complementary to the amplified oligo on the bead. Then, 1 μL of 10 μM forward primer was added, and the annealing process was repeated as initially described.

### 3. Polyacrylamide Gel Electrophoresis (PAGE) Analysis

To verify the bands of oligos obtained from the synthesis and selection process, PCR was conducted before PAGE analysis. Before amplification, the cycle threshold was measured using Luna® Universal qPCR Master Mix (New England Biolabs) and the CFX Connect Real-Time PCR Detection System (Bio-Rad) with the following protocol: (1) initial denaturation at 95° C. for 1 min, (2) denaturation at 95° C. for 15 sec, (3) extension at 60° C. for 30 sec and plate read, with steps 2-3 repeated for 35 cycles. Amplification was carried out through a saturation cycle using AccuPrime™ Taq DNA Polymerase. The amplicon was electrophoresed on an 8% polyacrylamide denaturing gel containing 7M urea at 200V for 30 minutes. The gel was stained with SYBR Gold (Thermo Scientific™) and imaged using the Invitrogen iBright FL1500 Imaging System (Thermo Scientific™) to confirm the presence of bands.

### 4. Data Encoding and Decoding Process

A total of 96.88 KB of Musical Instrument Digital Interface (MIDI) files were encoded into a DNA sequence diversity of 12,000 using DNA fountain code and synthesized by Twist Bioscience. For a targeted subset replacement, 766 bytes of the MIDI file were encoded into 80 DNA sequences, also synthesized by Twist Bioscience. The data were encoded within 156 nt of the 200 nt synthesized oligos. Following selection, the decoding process was performed from the raw data obtained through sequencing. Error correction was applied during the decoding back to MIDI files for sequences that did not fully align with the reference due to sequencing errors. This error correction was facilitated by a Reed-Solomon (RS) code of 2-10 nt incorporated into the 156 nt during the encoding process.

### 5. NGS Data Analysis

Raw FASTQ files were obtained from NGS sequencing. The paired-end reads were merged using FLASH for further analysis. The merged reads were filtered using FASTP to ensure a quality score above 30 and aligned with reference sequences using BWA. The SAM files of aligned reads were converted into BAM files utilizing SAMtools (http://www.htslib.org/doc/samtools.html), and a text file containing sequences and their respective read counts is obtained based on the BAM files.

### Results

### 1. Demonstration of Multiple Modes of Oligo Subset Selection Using Five Distinct Oligos

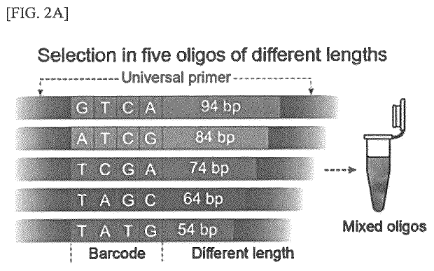

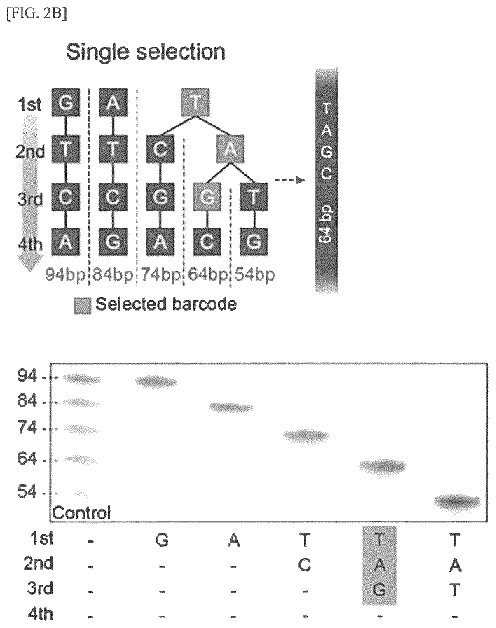

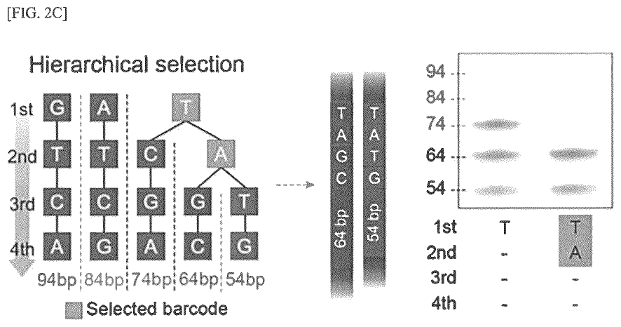

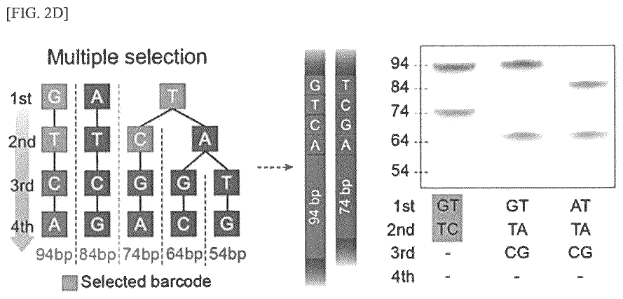

To validate the proposed method, five oligos with distinct barcodes and varying lengths (54, 64, 74, 84, and 94 bp) were designed to facilitate differentiation via electrophoresis (FIG. 2). Each oligo contained an identical universal primer sequence of 20 nt at one end, followed by a barcode region. Theoretically, a 2 nt barcode is sufficient to differentiate and encode the five subsets. However, to demonstrate the capability of hierarchical and simultaneous selection of multiple barcodes, we assigned a 4 nt barcode (FIG. 2A). Oligos were mixed in equal molar proportions and selected using a combination of reversible and irreversible terminators corresponding to the target barcode. For demonstrations, single, hierarchical, and multiple subset selections are conducted. For the selection of single subsets, the synthesis and selection processes were continued until the barcodes of each subset differed from one another (FIG. 2B). The oligos that were denatured and analyzed by polyacrylamide gel electrophoresis demonstrated varying band lengths depending on the selection, in contrast to the control, where all five oligo bands were visible. The capability of hierarchical selection of all “files” within a “folder” simultaneously by targeting a top-level barcode sequence was then examined (FIG. 2C). Selecting the first barcode, T, resulted in bands of 74, 64, and 54 bp oligos. Extending selection to the second barcode A isolated files under the folder labeled A, bands for the 64 and 54 bp oligos were acquired. In addition, this method was applied to select subsets with multiple barcodes simultaneously (FIG. 2D). For example, by employing reversible terminators for G and T and irreversible terminators for A and C, simultaneous selection to the barcodes G and T was achieved. Subsequent selection of the second barcodes T and C enabled specific selection of the 94 bp and 74 bp oligos in the sub-layer of T. The simultaneous selection of multiple barcodes was feasible regardless of the barcode sequence or hierarchical position. Thus, the proposed synthesis and selection method can be applied for oligo subset selection in a highly programmable manner, thereby enabling multiple modes of subset selection.

### 2. Subset Selection in Hierarchically Encoded Complex Oligo Libraries

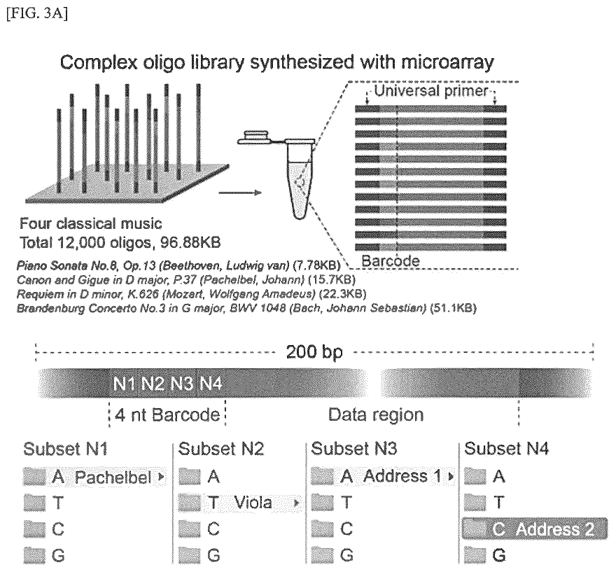

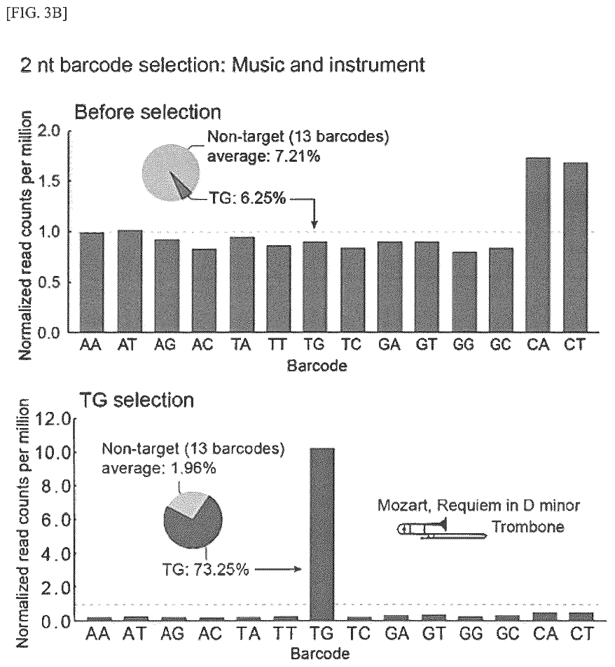

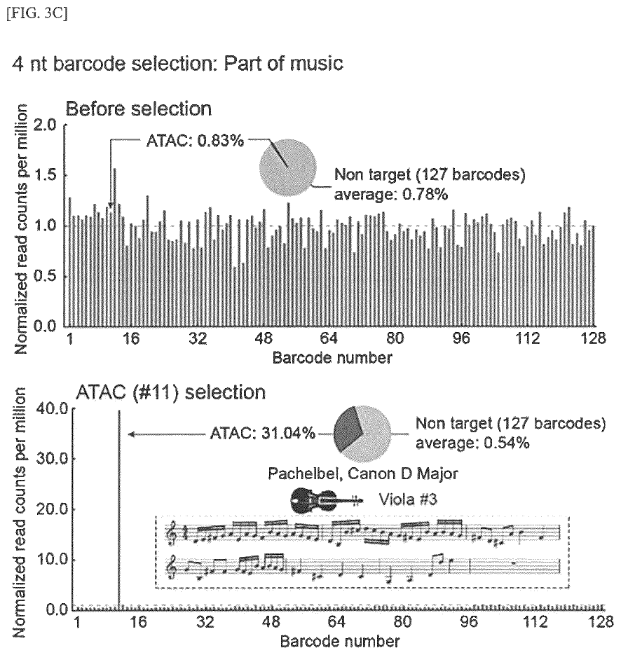

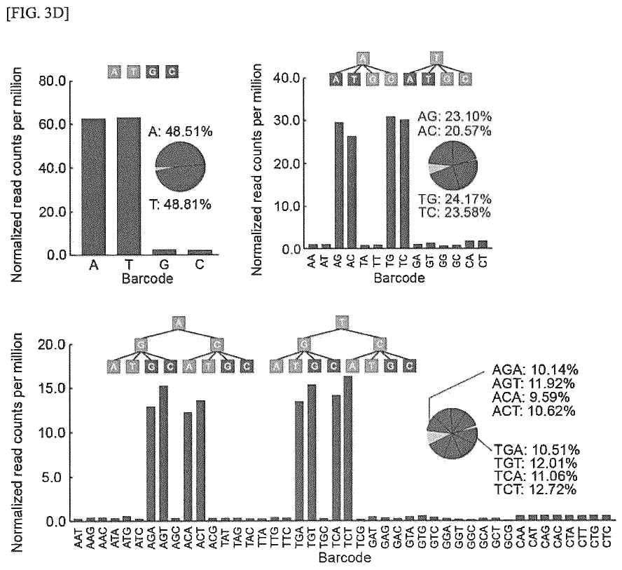

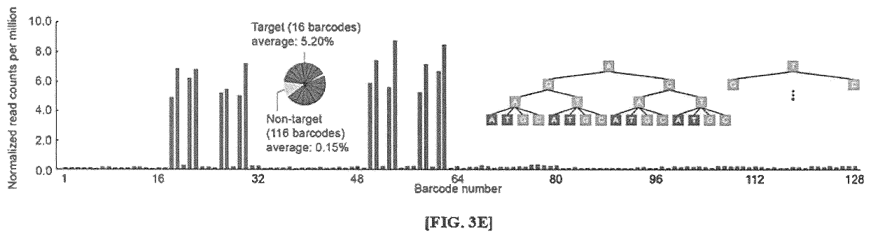

To validate the scalability of the subset selection via synthesis and selection, we synthesized a complex oligo library that encodes digital data and selected various target subsets (FIG. 3). The data of four classical music pieces were encoded into a library composed of 12,000 oligos of 200 nt, each differentiated by unique 4 nt barcodes (FIG. 3A). We designed a hierarchical barcode structure: the first sequence of barcodes identified the musical piece, the second specified the instrument, and the third and fourth sequences denoted sections of the music. For instance, the first sequence ‘A’ represents Pachelbel's Canon in D Major, and the second sequence ‘T’ denotes the viola part, while subsequent sequences denote the sections. The 4 nt barcodes used in the experiments accounted for 128 subsets, with each subset assigned between 60 and 130 oligos. Based on the designed barcode structure, we selected various hierarchical levels of subsets from the complex oligo library (FIG. 3B, and FIG. 3C). First, 2 nt level subsets were selected from a group of 14 subsets. We targeted the trombone part of Mozart's Requiem in D minor and analyzed the enrichment results by normalizing and plotting NGS read counts before and after selection (FIG. 3B). The read count of the oligo pool before selection indicated that the values for each barcode were aligned with the designed ratio. The CG and CC barcodes were intentionally excluded, resulting in a two-fold prevalence of the CA and CT barcodes. After the TG barcode was selected, all barcodes except TG fell below the original ratio, while TG soared to more than 10-fold from 6.25% to 73.25%. The efficiency of target enrichment was also maintained in longer barcode-based selection. Next, we identified a larger number of 128 subsets using a 4 nt barcode (FIG. 3C). We selected the ATAC barcode to access a file comprising the third section of Pachelbel's Canon in D Major Viola. The ATAC barcode presence in the library was 0.83% before selection, but it increased to 31.04% after selection, increasing by 37.4 times. As a result, synthesis and selection-based selection could enrich hierarchically structured oligo subsets 10- to 30-fold.

Additionally, we demonstrated multiplexed selection in a complex oligo library (FIG. 3D, and FIG. 3E). In every synthesis and selection cycle, two barcodes were simultaneously selected. The selection of target bases generally resulted in enrichment levels more than 34 times those of non-target barcodes, demonstrating consistent enrichment across all barcode lengths without significant bias. The slight differences in percentages were influenced by the designed size differences between each subset. The advantages of synthesis- and selection-based selection demonstrate numerous selection modes in complex oligo libraries with reliable efficiency.

### 3. Selection Efficiency of Multiple Cycles of Selection and Rare Population of Oligo Subset

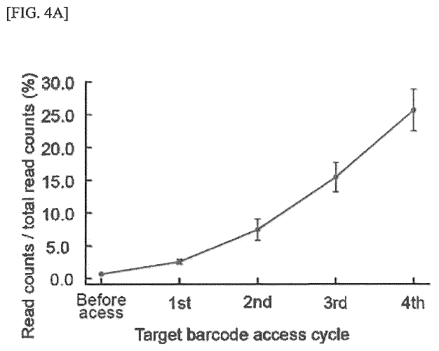

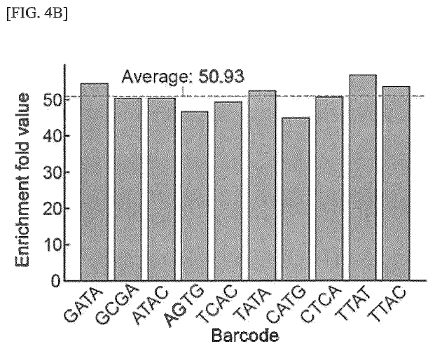

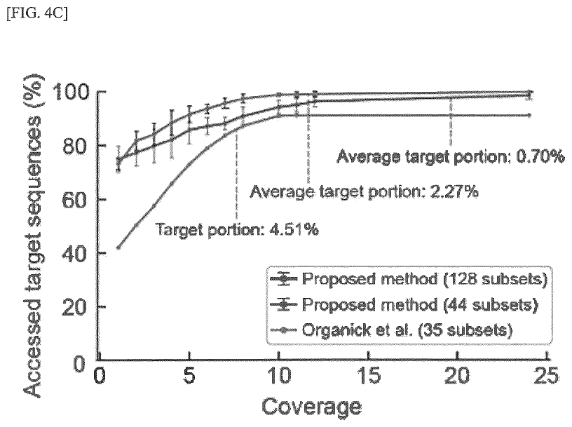

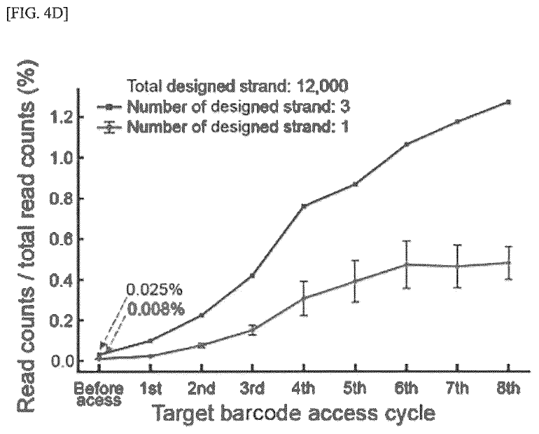

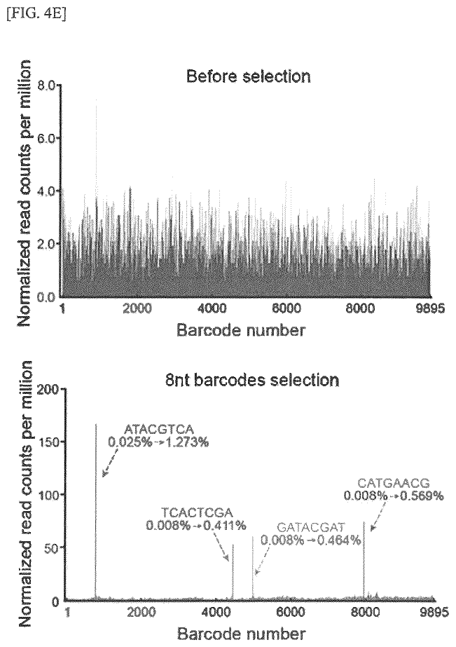

For an in-depth analysis of oligo subset selection from a complex oligo library, the selection efficiency of the 4 nt barcode was investigated at each step of cyclic DNA synthesis (FIG. 4). The oligo subset of 4 nt barcodes consists of 60, 80, and 100 distinct oligo designs from the oligo library, with a diversity of 12,000, corresponding to theoretical ratios of 0.5%, 0.67%, and 0.83%, respectively. Before selection, the average ratio of the subset was 0.68%, which increased with each cycle as the subset was continuously specified throughout the selection process. After four cycles of selection, the ratio reached 25.6%, which was a 37.6-fold increase due to selection. (FIG. 4A). We also measured the enrichment fold (EF) for each oligo subset selected using 10 distinct 4 nt barcodes. The EF can be calculated using the mathematical equation of the read fraction after selection (RFA) and the read fraction before selection (RFB):

$$EF = \frac{RFA ( 1 - RFB )}{RFB ( 1 - RFA )}$$

The average EF value was 50.93 (FIG. 4B), and because there was no significant bias for various barcodes, we believe that complexity could be increased by utilizing barcodes up to the theoretical level (4^{N} with an N-nt region). In addition, the recovery rate of the oligo subset of the selected barcode according to sequencing coverage was measured and compared with that of the PCR-based method, confirming that the proposed method is less prone to molecule loss after selection (FIG. 4C). For comparison, the selection of a single subset with a 4 nt barcode, and multiple subsets with a 3 nt barcode were analyzed. As a result, the PCR-based selection loses approximately 9.1% of the subset no matter how much the sequencing coverage; however, our method only causes a loss of less than 2% for single subset selection and 1% for multiple subset selection. Although the original average target portion in the oligo library was smaller than that in the PCR-based selection, it demonstrated a high recovery rate of the target DNA subset, and successful data recovery.

By performing barcode selection beyond 4 nt barcode selection up to 8 nt, we also checked whether it was possible to recover the rare oligo subset within an oligo library consists of three distinct sequences out of a diversity of 12,000, which represents a theoretical ratio of 0.025% (FIG. 4D). Additionally, to verify the possibility of more severe cases, we selected a library with a theoretical ratio of 0.008%, that consists of one sequence out of a diversity of 12,000. For a total of four 8 nt barcodes, the selection efficiency was investigated at each step of cyclic DNA synthesis. An increasing trend in efficiency was observed for each cycle as the subset was continuously specified throughout the selection process. After eight cycles of selection, the ratios reached 1.27% and 0.48% for three distinct oligo designs and one oligo design, respectively. This represents a 42.4-fold and 48.1-fold increase in counts, respectively, due to the selection process. When the normalized read count per million before and after the selection of an 8 bp barcode was measured, we confirmed that the ratio of the target barcode improved after selection compared with that before selection (FIG. 4E). This value is significantly higher than that of non-target barcodes, and it is possible to effectively access the data encoded in the corresponding DNA sequences.

### 4. Targeted Subset Replacement in a Hierarchically Structured Oligo Library

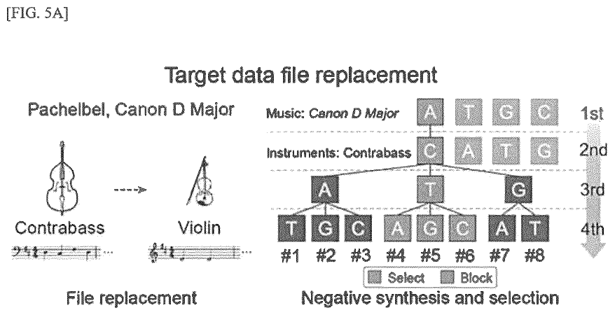

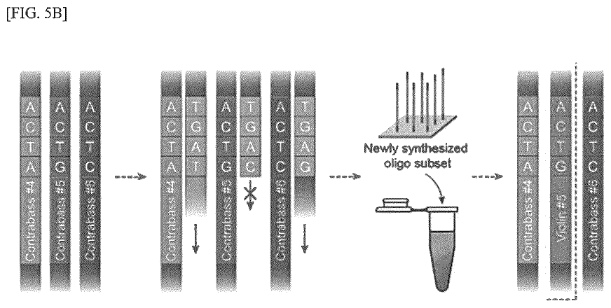

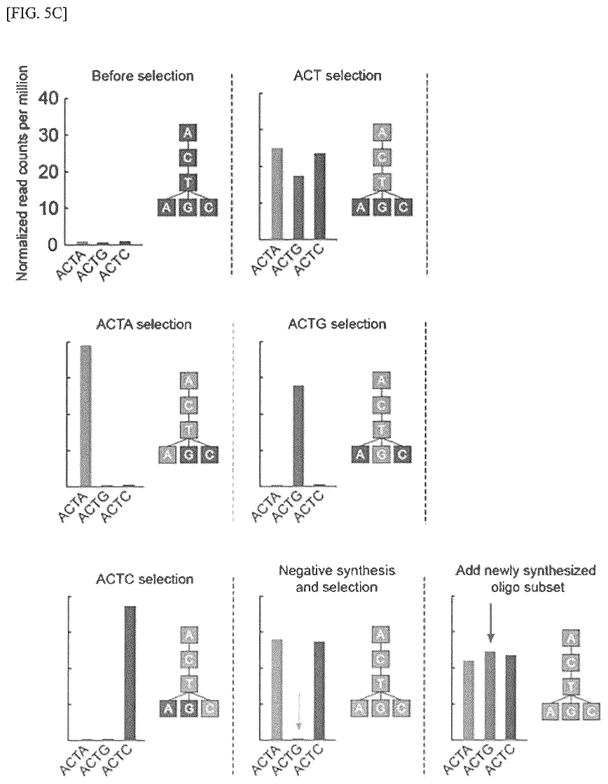

Moreover, we verified the possibility of replacing target subsets without affecting the original library by negative synthesis and selection followed by new subsets addition (FIG. 5). The aim of this experiment is to replace a contrabass file with a violin file. For file replacement, we selected up to the third directory containing the target file and then blocked the target file while allowing selection of the others (FIG. 5A). Blocking specific barcodes while allowing the selection of others prevents the elongation of subsets designed to be deleted and enables the elongation of other subsets. After elongation, the subset that failed to recover its original length was removed. The file replacement process was completed by adding a newly synthesized oligo subset encoded with the replacement data (FIG. 5B). To verify whether file replacement was successful, we plotted the NGS results of the read counts using barcodes (FIG. 5C). If the negative synthesis and selection processes worked properly, only the reads for the ACTG barcode were reduced from the ACT selection results. Indeed, the reads for ACTG decreased, whereas those for ACTA and ACTC increased. To determine whether there was a difference in efficiency between the original selection approach and negative synthesis and selection, we compared the selection results for the ACTA, ACTG, and ACTC barcodes. The reads for unselected barcodes appeared to be comparable across both methods. After adding a newly synthesized oligo library following negative synthesis and selection, we observed reads from the new library containing the ACTG barcode, confirming that the file replacement was successful.

### Discussion

In this study, we propose a synthesis and selection-based oligo subset selection method that distinguishes target molecules from a complex oligo library by single-nucleotide resolution with high efficiency and programmability. To the best of our knowledge, this is the first attempt at selecting oligo subsets from a complex library that does not rely on selective hybridization. While conventional methods, such as PCR and hybridization-based capture, typically require barcodes of at least 40 nt, regardless of the library's complexity, our proposed method can encode N distinct oligo subsets with only approximately [log₄N] nt barcode regions. For instance, 14 to 128 types of subsets were encoded with only 2 to 4 nt barcode regions, which is less than 2% of the total oligo length. This is a substantial improvement, considering that previous studies required approximately 15%-25% of the total oligo length. Furthermore, there are additional restrictions in primer sequence design to minimize secondary structure and crosstalk between distinct barcodes, and approximately 6,000 subsets were designed with 40 nt barcodes. By contrast, proposed method allows programmable barcode design in lengths that can be adjusted based on the number of subsets, and 47,088 subsets were encoded with 8 nt barcodes—approximately 39.2 times more barcode per nt than that of PCR-based methods. Furthermore, 415 billion subsets can theoretically be encoded using a 20 nt barcode.

We have enriched the target oligo subsets with two synthesis and selection cycles from 6.25% to 73.25%, whereas that of other subsets was decreased to 1.96% or 37.4-fold. The increased target subset ratio enabled decoding of all target subsets within the oligo library with reduced sequencing depth. A possible reason for limited enrichment is the nucleotide coupling efficiency of the polymerase, and we believe that enrichment can be improved if the performance of the polymerase is optimized. Although a synthesis and selection require universal primers, these can be attached through blunt-end ligation, which along with reduced barcode regions, can lower both synthesis and sequencing costs. Finally, our approach significantly enhances the utility of complex oligo libraries, which are crucial for applications in gene synthesis, perturbation screening, and especially DNA data storage, and can be further applied to the identification of various targets of interest with high sequence similarity from complex biological pools.

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

What is claimed is:

1. A method for selecting the target nucleic acid comprising a step of:
(a) identifying a part of the nucleotide sequence of the target nucleic acid as XₙXₙ₊₁;
(b) treating a mixture composed of two or more types of base units to target nucleic acid Xₙ;
(c) removing the bases mixture from step (b); and
(d) recognizing the base units bound to the target nucleic acid in step (b),
wherein n is a natural number.

2. The method according to claim 1, wherein the mixture composed of two or more types of base units in step (b) is a mixture of bases with reversible terminators and bases with irreversible terminators.

3. The method according to claim 1, wherein the mixture composed of two or more types of base units in step (b) is a mixture of unlabeled dNTPs and bases with reversible terminators.

4. The method according to claim 1, wherein the mixture composed of two or more types of base units in step (b) is a mixture of unlabeled dNTPs and bases with irreversible terminators.

5. The method according to claim 1, wherein the mixture composed of two or more types of base units in step (b) is a mixture of unlabeled dNTPs, bases with reversible terminators and bases with irreversible terminators.

6. The method according to claim 1, further comprising, after step (d), a step of (e) polymerizing bases to bind only to the target nucleic acid using a polymerase.

7. A method for selecting the target nucleic acid comprising a step of:
(a) identifying a part of the nucleotide sequence of the target nucleic acid as XₙXₙ₊₁;
(b) treating a mixture composed of complementary bases with reversible terminators and non-complementary bases with irreversible terminators to target nucleic acid Xₙ;
(c) removing the bases mixture from step (b); and,
(d) removing the blocker of the reversible terminator from step (b),
wherein n is a natural number.

8. The method according to claim 7, further comprising, after step (d):
(e) treating a mixture composed of complementary bases with reversible terminators and non-complementary bases with irreversible terminators to target nucleic acid Xₙ₊₁;
(f) removing the bases mixture from step (e); and,
(g) removing the blocker of the reversible terminator from step (e).

9. The method according to claim 7, wherein the reversible terminator is selected from the group consisting of an azidomethyl moiety, an allyl moiety, and a nitrobenzyl moiety.

10. The method according to claim 9, when the reversible terminator is an azidomethyl moiety, the removing the blocker of the reversible terminator in step (d) is performed using tris(2-carboxyethyl) phosphine.

11. The method according to claim 9, when the reversible terminator is an allyl moiety, the removing the blocker of the reversible terminator in step (d) is performed using sodium tetrachloropalladate, or sodium triphenylphosphine trisulfonate.

12. The method according to claim 9, when the reversible terminator is a nitrobenzyl moiety, the removing the blocker of the reversible terminator in step (d) is performed by laser irradiation of 345 to 365 nm.

13. The method according to claim 7, wherein the bases with irreversible terminators are a dideoxynucleotide (ddNTP).

14. The method according to claim 7, wherein the base is selected from the group consisting of adenine, thymine, cytosine, guanine, isoguanine, isocytosine, 2-amino-6-(2-thienyl)purine, pyridine-2-one, pyrrole-2-carbaldehyde, 7-(2-thienyl)imidazo[4,5-b]pyridine, 2,6-dimethyl-2H-isoquinoline-1-thione, 2-Methoxy-3-methylnaphthalene, 2-amino-imidazo[1,2-a]-1,3,5-triazin-4(8H)one, 6-amino-5-nitro-2(1H)-pyridone, 7-(2,2′-bithien-5-yl)-imidazo[4,5-b]pyridine, 4-[3-(6-aminohexanamido)-1-propynyl]-2-nitropyrrole, and inosine.

15. The method according to claim 7, further comprising, after step (a), the step of (a-1) adding a primer that recognizes the polynucleotide sequence of the target nucleic acid.

16. A method for elongating the target nucleic acid comprising a step of:
(a) selecting the target nucleic acid according to the method of claim 7; and
(b) repeatedly performing the method of claim 8.

17. A composition for selecting a target nucleic acid, comprising dATP with reversible terminators, ddTTP, ddCTP, and ddGTP,
wherein the reversible terminator is selected from the group consisting of an azidomethyl moiety, an allyl moiety, and a nitrobenzyl moiety.

18. A composition for selecting a target nucleic acid, comprising dTTP with reversible terminators, ddATP, ddCTP, and ddGTP,
wherein the reversible terminator is selected from the group consisting of an azidomethyl moiety, an allyl moiety, and a nitrobenzyl moiety.

19. A composition for selecting a target nucleic acid, comprising dCTP with reversible terminators, ddATP, ddTTP, and ddGTP,
wherein the reversible terminator is selected from the group consisting of an azidomethyl moiety, an allyl moiety, and a nitrobenzyl moiety.

20. A composition for selecting a target nucleic acid, comprising dGTP with reversible terminators, ddATP, ddTTP, and ddCTP,
wherein the reversible terminator is selected from the group consisting of an azidomethyl moiety, an allyl moiety, and a nitrobenzyl moiety.

21. A kit for selecting a target nucleic acid, comprising any one of the compositions of claims 17 to 20.


SEQUENCE LISTING (7 sequences)

SEQ ID NO: 1 - DNA, 33 bp, synthetic construct
  source 1..33: mol_type = other DNA
acactctttccctacacgacgctcttccgatct

SEQ ID NO: 2 - DNA, 34 bp, synthetic construct
  source 1..34: mol_type = other DNA
gtgactggagttcagacgtgtgctcttccgatct

SEQ ID NO: 3 - DNA, 54 bp, synthetic construct
  source 1..54: mol_type = other DNA
acacgacgctcttccgatctatacgtactgagacagatcggaagagcacacgtc

SEQ ID NO: 4 - DNA, 64 bp, synthetic construct
  source 1..64: mol_type = other DNA
acacgacgctcttccgatctatcggtactgagaccagatctgcgagatcggaagagcacacgtc

SEQ ID NO: 5 - DNA, 74 bp, synthetic construct
  source 1..74: mol_type = other DNA
acacgacgctcttccgatctagctgtactgagaccagatctgcgagacggctgtagatcggaagagcacacgtc

SEQ ID NO: 6 - DNA, 84 bp, synthetic construct
  source 1..84: mol_type = other DNA
acacgacgctcttccgatcttagcgtactgagaccagatctgcgagacggctgtttaacagattagatcggaagagcacacgtc

SEQ ID NO: 7 - DNA, 84 bp, synthetic construct; identical to SEQ ID NO: 6
  source 1..84: mol_type = other DNA